(19) United States
(12) Patent Application Publication
(10) Pub. No.: US 2004/0073222 A1
(43) Pub. Date: Apr. 15, 2004

(54) Ligament fixing system

(76) Inventor: Tomoaki Koseki, Tokyo (JP)

Correspondence Address: Tomoaki Koseki, 7 3 Komagome 7 Chome, Toshima Ku, Tokyo, 170-0003, JP

(21) Appl. No.: 10/182,285

(22) PCT Filed: Nov. 28, 2001
(86) PCT No.: PCT/JP01/10359

(30) Foreign Application Priority Data
Nov. 28, 2000 (JP) 2000-403551

(51) Int. Cl. A61B 17/56

(52) U.S. Cl. 606/75

(57) ABSTRACT

An inexpensive ligament fixing system capable of providing an efficient fixing force and accurately driving a staple with less invasion, comprising a flat-shaped plate having a plurality of drilled holes and curved bottom and upper surfaces and formed by casting and a cross bar used to allow a parallelism, to be checked, wherein the plate is fixed to a ligament and the folded-up ligament is further fixed with the staple, the tips of the leg parts of the staple are moved from the centerline to the inside, a cut-in is provided on the insides of the leg parts, the cross sections of the leg parts are formed in a polyhedron, the plate is pressed and driven by a driving device having a driving structure by a thrust bar is used.

### FIELD OF THE INVENTION

This invention is related to the fixing system combining a staple and a plate to fix a ligament on a bone surface, the system of which is utilized during a ligament repair operation.

### PRIOR ART

A common method to fix a ligament, is the so called Double Stapling method in which the ligament is folded up in the opposite direction after fixing the ligament with one staple, and the second staple is fixed on top of the folded-up ligament in proximity to the first staple. Also, to provide stronger fixing force, there are cases where a multiple number of staples are fixed within some space at one end of the same ligament.

The sharp tip end of the staple is on the center line of the staple leg part. Furthermore, the cross-section of the staple leg part is either round or square.

A driving device to fix the staple has the structure of either holding the horizontal part of the staple or picking and then holding the middle part of the staple leg part, and then the latter half of the staple is fixed.

The staple and the driving device are manufactured with cut-shave processing method.

### SUMMARY OF THE INVENTION

The conventional double stapling method catches the tissue with the two legs of the staple fixed on the tissue, and therefore could easily produce staple site pain (pain caused by the staples). Also, as the two staples generate some volume sunk within the tissue, there is more invasiveness to the patient.

In cases of ACL (anterior cross ligament) and such, the presumption is that the staples will be removed after the ligament is rebuilt, but with the double stapling method, the bone tissue is often destroyed at the time of staple removal as the staple leg parts catch the bone tissue.

When fixing the ligament, the fixing force is strongest when the staple is driven perpendicularly to the longitude direction of the ligament. When fixing an end of a ligament with a multiple number of staples, the perpendicular angle is measured at a rough estimate wherein a certain angle of the staples is difficult to be maintained, thus difficult to obtain efficient fixing force.

The bone surface is in a column form and is also slippery. In most cases the staples tend to have the leg parts to widen when driven into the bone, and staples with widened legs are easily loosened.

When staples are fixed in situations with the staple's horizontal parts or its leg parts caught part of the way, the staple ends easily slip on the bone surface, the leg parts of the staple widens easily, thus the driving angle cannot be maintained in a certain angle, a situation which hinders stable driving of the staples.

The cut-shave processing manufacturing method takes much time and labor, and is also costly. Also products made per the manufacturing method have difficulties in coping with complex curved surface.

To solve the above issues, two staples are not driven in a row as in the Double Stapling method, but this invention makes it possible to realize less burden on the bone and also obtain sufficient fixing force, by combining a staple and a plate. The bottom side of the flat plate has a multiple number of spikes to temporarily fix the pulled up ligament on the bone surface, and there are holes in the central part along the periphery. A U-shaped staple has the exact size where the horizontal part can be inserted into the holes on the plate, the pulled up ligament is folded up in the opposite direction, and so when the staple is fastened, the two legs of the staple approach the bone surface through the hole part of the plate, the bottom part of the horizontal part of the staple presses the ligament on the bone surface, and thus fixes the ligament.

For parts where the burden is larger, the type to be used will be the one in which the plate has holes along the central part the holes made in parallel to the longitude direction of the plate, thus the ligament can be fixed with more force using a multiple number of staples.

By realizing the plate bottom to have a curved surface, the plate bottom has closer contact on the bone surface, which prevents the staples to slip on the bone surface when fixing. Also, as the plate top part which touches the ligament surface has its corners in a round form with a smooth curve, the ligament is not damaged and so there will be no uncomfortable feeling on the body surface after the staple stitches sink within the tissue.

When the same end of a ligament is to be fixed with a multiple number of staples and plates, an X shape cross bar is utilized. When pins at the apexes of the four corners (of the X shape) are attached in the plates, it can be confirmed that each plate is placed in parallel to each other. By achieving this, the staples' angle can be maintained in a certain angle, and efficient fixing force can be obtained.

By having the sharp ends of the two legs of the staple to be drawn inwards from the center line of the leg part, the widening of the staple's legs at the moment of fixing can be prevented. Also, the same effect can be obtained by providing one or a multiple number of cuts in the inside of both legs at the center of the legs.

By realizing the cross section of the staple legs to be in a polygon form with more sides than a pentagon, and the line connecting each apex to be not in a straight line but in a curve towards the inner side of the legs, the contact area in the vertical direction within the bone is decreased and therefore there will be less resistance at the moment of staple fixing. When the staple is fixed, the staple will go inside the tissue vertically in relation to the bone surface, but after the fixing, the staple's releasing force will work sideways, as the ligament is fixed onto the bone surface horizontally. When the staple leg part is in a curve form curving inward, the curve will increase the contact area with the bone tissue in contrary to the staples'releasing force, and therefore the resistance will be enlarged. At the point of removal of the staples, the staples are pulled out once again in vertical direction, therefore the resistance will be small.

The driving device with which the above staples and plates are driven, will have a concave formed tips to enable the plates to fit, to facilitate the plates to be fixed easily on the bone surface once the grip latter end is driven. There will be an oblong window on the foreside of the tip part for inserting the staple, and the staple will be placed inside from that window. The staple tips will touch the bone surface, but the leg parts are all covered with a cover. The inside of the grip connected to the tips are in cylindrical form, and a slide bar is inserted inside the cylinder and the latter end of the slide bar is driven, thus enabling the staple at the tip end to be driven and fixed into the bone. The staple will accurately go inside the fixed plate holes, the leg parts are covered so the direction and the angle are stable. The staple's two leg will not be widened.

A wax mould of the said staple, plate, driving device shall be made, the peripheral of the model is hardened with fireproof material, then heated and the wax will melt away, then metal liquid is poured within the made space then cooled down. This manufacturing method utilizing a mould to shape the form will enable affordable products with stable form. With this method it is easy to produce complex curves.

### DETAILED dESCRIPTION OF THE PREFERRED EMBODIMENTS

Hereafter, explanation of the realization of this invention is made in reference to drawings. FIG. 1 shows the fixing system of combining Staple 1 and Plate 2 according to claim 1. The bottom part of Plate 2 has Spikes 4 on the Plate's four corners, the Spikes temporarily fix Ligament 3 on the bone surface, and the central part of the plate has holes. Ligament 3 is folded up in the opposite direction from the originally pulled direction, and when Staple 1 is driven, the two Legs 5 of Staple 1 reach the bone surface through the hole parts of Plate 2, the bottom of the Horizontal part 6 of Staple 1 presses the Ligament 3 onto the bone surface, and will fix Ligament 3.

FIG. 2 shows the type according to claim 2, the type of which has two rows of holes in the plate center aligned along the longitudinal direction of the plate. By driving two staples, the ligament can be fixed with stronger fixing force.

FIG. 3 shows the type according to claim 3, the plate type of which has the plate bottom in a curve form to realize close contact with the bone surface, and also the plate top part to have all of its corners to be in a round form to enable the contact part with the ligament to be in a smooth curve. In this type, staples will not slip on the bone surface at the moment of driving the staples, thus the ligament is not damaged, and therefore there is less uncomfortable feeling on the body surface after the stitches sink within the tissue.

FIG. 4 shows the type according to claim 4, the type of which has an X form parallel maintenance device which enable a user of this device to confirm that each plate is in parallel to each other utilizing the X form Crossover 7 when fixing the end of Ligament 3 with a multiple number of Staples 1 and Plate 2. Pins 8 at the four apexes of the X form Crossover 7, the Pins of which to connect onto the plate, are arranged vertically to X form Crossover 7. By inserting the Pins 8 through the temporarily fixing holes 9 made on the two ends of one side of the Plate 2 in longitudinal line, the two Plates 2 are fixed and the parallel position of the two plates can be confirmed. After the parallel positioning of the two plates, Plates 2 can be fixed on the bone surface, the X form Crossover 7 is released, and Staple 1 is driven.

FIG. 5 shows the Staple which has the structure to avoid the widening of the two legs at the moment of driving the staple, the structure of which is realized by having the tip ends of the two legs to turn inward from the center part of the leg. The figure is in a 1 to 2 ratio.

FIG. 6 shows the staple with the structure of having cuts 10a, 10b, and 10c on the inside center part of the two legs to prevent the widening of the two legs at the moment of driving the staple. In this FIG., the three cuts 10a, 10b, and 10c are made. The legs have the cross section in polygon form according to claim 7, and the line connecting each end tip is not in straight line but in a curve turning toward the inside of the legs.

FIG. 7 shows the Driving device according to claim 8, the device of which is to drive the Staple 1 and Plate 2. The tip part is in a concave form to enable the Plate 1 to fit, so when the latter end of Grip 11 is driven, the Plate 1 can be fixed onto the bone surface easily. The fore part of the tip part has an oblong window 12 to insert Staple 1, and Staple 1 is placed through this window. The other side of this oblong window 12 is a wall. Below the oblong window 12, a small window is open, the window of which to confirm the position of the tip end of Staple 1. The tip end of Staple 1 touches the bone surface, but the Leg part 5 is covered with a cover. The inside of Grip 11 which is connected to the tip part is a cylindrical form, and the Slide bar 13 is inserted into this cylinder, the latter part of Slide bar 13 is driven to enable Staple 1 at the tip to be driven into the bone tissue. Slide bar 13 has a form wherein its cross section is made to be the same in its width and depth with Staple 1, and the latter part of Slide bar having a disk for driving. When the disk at the latter part of Slide bar 13 is driven, Staple 1 enters in the holes of the fixed Plate 2 accurately, and as the Legs 5 are covered with cover, the direction and the angle of the driving is stabilized. Also the two legs will not widen.

This invention is conducted as in the above explanation, and therefore has the following effects:

According to claim 1, there is less burden placed on the bone, and also provides a fixing system with sufficient fixing force. A plate with spikes are fixed on the bone surface, the inclination angle of the staple is controlled due to the fixing of the ligament inside the plate frames, and there is very small occurrence of staple site pain:

According to claim 2, it is possible to strongly fix the ligament:

According to claim 3, the staple does not slip at the bone surface at the moment of driving the staple. Also, the ligament is not damaged, and there is not much uncomfortable feeling at body surface after the stitches sink within the bone tissue:

According to claim 4, the staple direction can be maintained in a certain direction, and efficient fixing force can be obtained:

According to claim 5 and 6, it is possible to prevent the widening of the two legs of the staple at the moment of driving the staple:

According to claim 7, there is least resistance at the moment of driving the staple. On the other hand, there is more resistance again the staple's releasing force. At the time of staple removal, the resistance will be small once again:

According to claim 8, the plate can be fixed on the bone surface easily. The staple enters into the plate holes accurately, and the direction/angle are stable. The two legs of the staple will not widen:

According to claim 9, a product with stable form can be manufactured in mass, at an affordable expense. It is also easy to product complex curved surface.

### BRIEF dESCRIPTION OF THE DRAWINGS

FIG. 1 is an external drawing of the fixing system utilizing staple and plate.

FIG. 2 is an external drawing of the type with two holes on the plate.

FIG. 3 is an external drawing of the type with the bottom and the top part of the plate in a curved form.

FIG. 4 is an external drawing of the type utilizing a cross bar.

FIG. 5 is a plan view of the staple the tip ends of which are turning inward.

FIG. 6 is a plan view of a staple with cuts in the inside of the two legs, and the leg cross section in pentagon form.

FIG. 7 is a front view, side view and bottom view of the driving device.

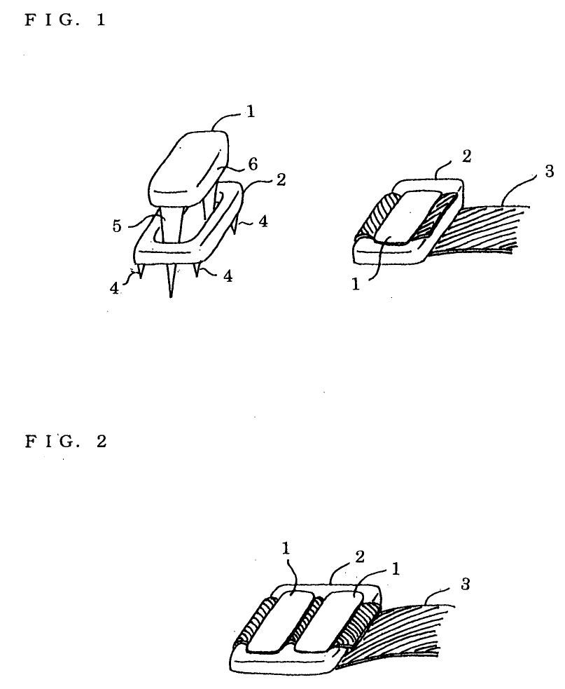

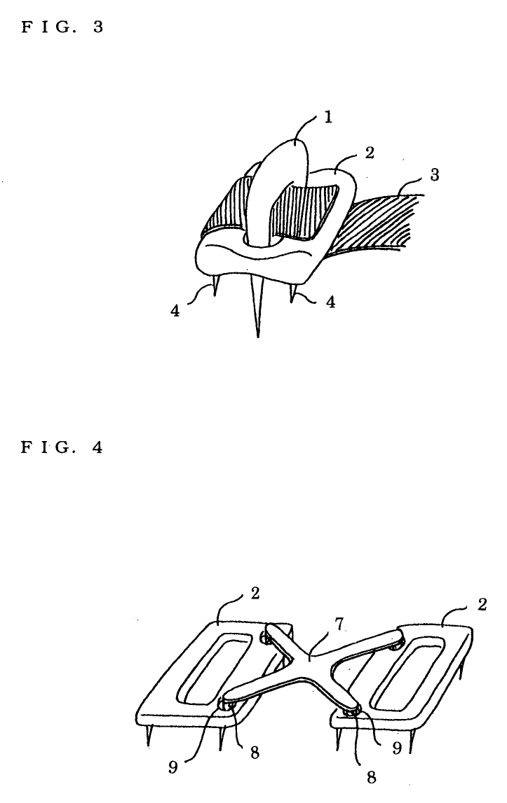

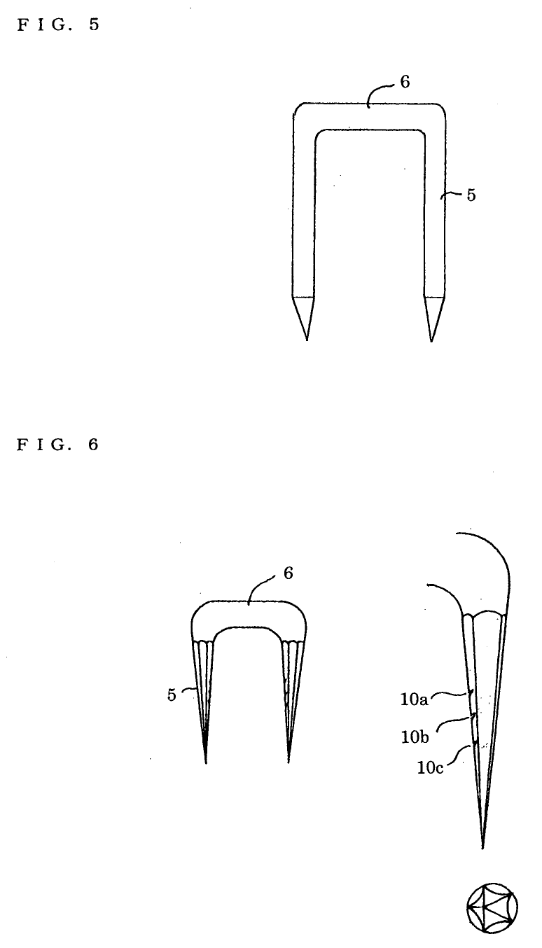

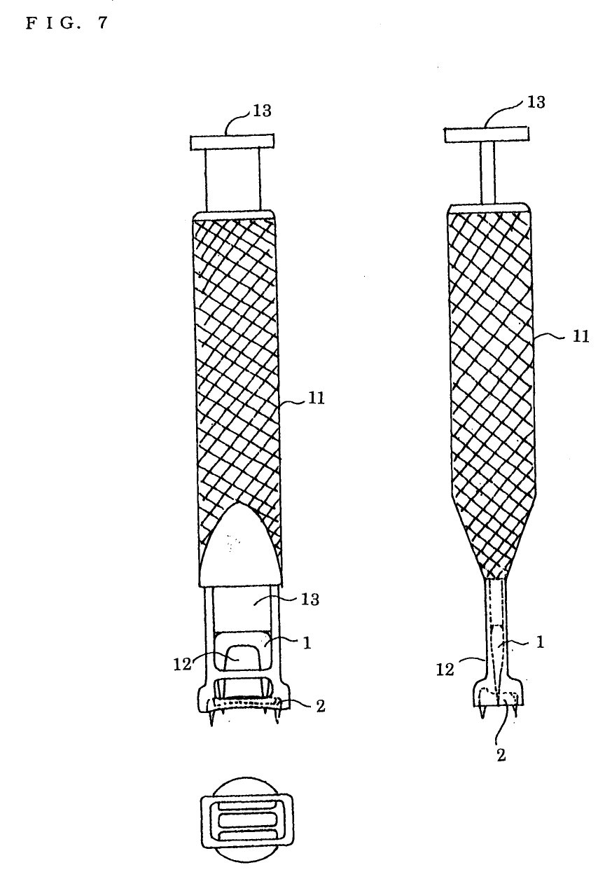

What is claimed is:

1. A fixing system combining staple and plate to fix the ligament on bone surface, a system of which is utilized in ligament repair operation, wherein a multiple number of spikes are attached on the base part of the flat plate, the spikes of which will temporarily fix the pulled ligament on bone surface, the center part of the plate having holes in line with the perimeter, the U shape staple being in a size to have the staple horizontal part to enter inside the plate hole, and when the ligament is folded up in the opposite direction from its pulled direction as though to cover the plate, and when the staple is driven above that plate, the two legs of the staple are driven on the bone surface through the plate holes, the staple base horizontal part will press the ligament on the bone surface, and thus the ligament is fixed.

2. Of the above explained fixing system of combining the staple and plate, a fixing system of combining staple and plate according to claim 1, the holes in the center part of the plate are in parallel to the longitude direction of the plate, and by driving staples which are in the size to enter inside each hole, ligament is fixed.

3. Of the above explained fixing system, a fixing system of staple and plates combined according to claims 1 and 2, where in the plate base is in a curved form to enable close contact of the plate and the bone surface, and also the plate top part having all the corners in a round form to realize the part that touches the ligament to be in a smooth curve.

4. Of the above fixing system of staple and plate combined, a fixing system combining staple and plate according to claims 1, 2 and 3, wherein the pins at the four corners of the X form cross bar, the pin of which to connect the plate, are perpendicular to the X form cross bar, and the pins are inserted in the temporarily fixing holes made at the two ends of the plate's longitudinal direction to enable the parallel positioning of the two plates and confirm the parallel position of the plate in relation to the other plate, when fixing an end of a ligament with a multiple number of staples and plates.

5. A staple to fix the ligament, utilized in a ligament repair operation, wherein the ligament fixing staple has the characteristic of which its sharp tip end of the two legs of the staple to be turned inward from the center, the staple will prevent the widening of the legs.

6. Ligament fixing staple utilized to fix ligament as in the said ligament repair operation, wherein the staple has the characteristic of having one or a multiple number of cuts in the inside of the center of the two legs, and thus prevent the widening of the legs at the moment of driving the staple.

7. The staple to fix ligament to be utilized in the above ligament repair system, the ligament fixing staple according to claims 5 and 6, with the characteristic of its two legs having polygon with multiple sides than pentagon, with the line connecting each tip end not in a straight line but in a curve turning toward the inside of the legs.

8. Of the fixing system combining the above staple and plate, the driving device to drive the staple and the plate, with the structure of the tip parts in a concave form so that the plate according to claims 1, 2, and 3 will fit, wherein there is a sideways window to insert the staple at the fore side of the tip end, the inside of the grip connecting to the tip end being in a cylindrical for, the slide bar is inserted inside the cylinder, and by driving the latter part of the slide bar the staple at the tip end is driven inside the bone.

9. The manufacturing method for the staple, plate, and driving device according to claims 1, 2, 3, 4, 5, 6, 7, and 8, wherein wax moulds of the staple, plate, and driving device are made, the peripheral is hardened with fireproof material, then the mould is melt after heating, then metal liquid is poured inside the made space and thus the model is made.
